(19) United States
(12) Patent Application Publication
(10) Pub. No.: US 2026/0187795 A1
(43) Pub. Date: Jul. 2, 2026

(54) Method and Apparatus for Anatomically Consistent Embeddings in Composition and Decomposition

(71) Applicants: Ziyu ZHOU, Shanghai (CN); Haozhe LUO, Zurich (CH); Mohammad Reza HOSSEINZADEH TAHER, Tempe, AZ (US); Jiaxuan PANG, Tempe, AZ (US); Jianming LIANG, Scottsdale, AZ (US)

(72) Inventors: Ziyu ZHOU, Shanghai (CN); Haozhe LUO, Zurich (CH); Mohammad Reza HOSSEINZADEH TAHER, Tempe, AZ (US); Jiaxuan PANG, Tempe, AZ (US); Jianming LIANG, Scottsdale, AZ (US)

(73) Assignee: Arizona Regents on behalf of Arizona State University, Scottsdale, AZ (US)

(21) Appl. No.: 19/427,881

(22) Filed: Dec. 19, 2025

Related U.S. Application Data
(60) Provisional application No. 63/740,011, filed on Dec. 30, 2024.

(51) Int. Cl. G06T 7/00; G16H 30/40

(52) U.S. Cl. CPC G06T 7/0012; G16H 30/40

(57) ABSTRACT

A self-supervised learning (SSL) model to learn an anatomically consistent embedding from unlabeled medical images receives a plurality of unlabeled medical images including consistent large/global and small/local anatomical structures, and divides each of the plurality of unlabeled medical images into a grid of non-overlapping patches. Two random crops are extracted from each of the plurality of unlabeled medical images, wherein each of the two random crops comprises a subset of the grid of non-overlapping patches and shares with the other of the two random crops a partially overlapping region of the grid of non-overlapping patches. A global consistency branch is executed that captures discriminative macro-structures in the plurality of unlabeled medical images by extracting global embeddings. A local consistency branch is concurrently executed that learns fine-grained local anatomical details in the plurality of unlabeled medical images via composition and decomposition operations.

### CLAIM OF PRIORITY

This application claims the benefit of U.S. Provisional Patent Application No. 63/740,011, filed Dec. 30, 2024, entitled “ACE: ANATOMICALLY CONSISTENT EMBEDDINGS IN COMPOSITION AND DECOMPOSITION”, the disclosure of which is incorporated by reference herein in its entirety.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of this document contains material which is subject to copyright protection. The copyright owner has no objection to the facsimile reproduction by anyone of the document as it appears in the Patent and Trademark Office patent records, but otherwise reserves all copyright rights whatsoever.

### TECHNICAL FIELD

Embodiments of the disclosure relate to self-supervised machine learning models and in particular a grid-wise image cropping of medical images to leverage the intrinsic properties of compositionality and decompositionality of medical images, bridging a semantic gap from high-level pathologies to low-level tissue anomalies, and providing a new self-supervised machine learning model for medical imaging.

### BACKGROUND

Considering that expert labeling is costly and labor intensive in medical imaging, self-supervised learning has become a key approach towards annotation efficiency. Recent SSL methods, mainly designed for photographic images, do not fully exploit the inherent characteristics of medical images. Medical images (e.g., chest X-rays, fundus photography), unlike photographic images with target objects typically at the center on varying backgrounds, showcase consistent global (lung, heart) and local (clavicle, bronchus) anatomical structures as illustrated in FIG. 1A, resulting from standardized imaging protocols. In addition, from an anatomical point of view, organs or tissues are decomposable, such as the left lung consisting of the superior lobe and the inferior lobe shown in FIG. 1B. To utilize these medical priors it is hypothesized that simultaneously learning from global and local consistencies via composition and decomposition can equip the model to understand the anatomy, thereby offering strong transferability.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1A is a chest X-ray that contains various large (global) and small (local) anatomical patterns, including the right/left lung, heart, spinous processes, clavicle, mainstem bronchus, and the osseous structures of the thorax, which can be utilized for learning global and local embeddings in anatomy.

FIG. 1B illustrates the hierarchical nature of anatomy (e.g., the left lung has two lobes, the superior lobe x and the inferior lobe y), calls for anatomical representation with compositionality where the embedding of the whole patch should be the sum of the embeddings of each part.

FIG. 2 depicts a functional block diagram according to the disclosed embodiments.

FIG. 3A illustrates that the disclosed embodiments preserve the compositionality of anatomical structures in a learned embedding space.

FIG. 3B illustrates that a distribution according to the disclosed embodiments is narrower and taller compared with DINO, DropPos and SelfPatch, with the mean similarity between embeddings of patches and their compositional parts closer to 1.

FIG. 4A illustrates how the disclosed embodiments preserve the decompositionality of anatomical structures in its learned embedding space.

FIG. 4B shows the disclosed embodiments outperform SSL baselines by a large margin, achieving a high accuracy of 89.01%, which is 30% higher than the second-best baseline.

FIG. 5A illustrates the disclosed embodiments capture of semantics-rich features in its learned embedding space.

FIG. 5B shows the disclosed embodiments achieve higher retrieval accuracy compared with other SSL baselines.

FIG. 6 illustrates that the disclosed embodiments reflect locality of anatomical structures in the learned embedding space as an emergent property, in particular, ACE, unlike the SSL baselines, distinguishes different anatomical structures in its embedding space while keeping identical anatomical structures across patients close to each other.

FIG. 7A demonstrates the disclosed embodiments' ability to accurately identify anatomical landmarks across different patients, including Zero-shot predictions and ground truth of selected query landmarks.

FIG. 7B shows quantitative analysis with a low prediction error of 61 pixels in 10242 images, highlighting the robustness of the ACE model features in consistent cross-patient anatomical identification.

FIGS. 8A and 8B demonstrate the disclosed embodiments superior performance in limited data regimes; as seen in both heart segmentation and pneumothorax classification tasks, ACE surpasses SSL baselines (DINO and POPAR), particularly in few-shot transfer settings.

FIG. 9 illustrates ablations on the learning objects, in which adding the losses improves performance on classification and segmentation.

FIG. 10A illustrates a comparison of ACE with other baselines, in which AUC scores are shown on the EyePACS diabetic retinopathy classification dataset.

FIG. 10B illustrates ACE's unsupervised landmark correspondence for paired left and rights fundus image on the FIRE dataset, in which the rounds on query and key images are paired ground-truth landmarks, and the crosses on key images are predicted ones.

FIG. 11 presents Table 1, which shows the ACE model with the ViT-B backbone delivers competitive or superior performance compared with baselines with the same backbone, including DINO, SelfPatch, and DropPos.

FIG. 12 includes a proposed pseudo-code implementation of local consistency in accordance with the disclosed embodiments.

FIGS. 13A, 13B and 13C illustrate how embodiments of the disclose reflect the symmetry of anatomical structures in the learned embedding space as an emergent property, wherein the embodiments provide mirrored embeddings for mirrored anatomical structures (e.g., the right and left clavicles, and the right and left rib 5).

FIGS. 14A, 14B and 14C demonstrate the ability of the disclosed embodiments to boost downstream key point detection tasks, in which 7 key points are chosen for fine-tuning (FIG. 14A); the inference detection image where the dots are prediction while the crosses are the ground truth situating at the center of the heatmap (FIG. 14B); and comparison between ACE and other pretrained baselines and the lower pixel error better detection performance (FIG. 14C).

FIG. 15 provides a visualization of Grad-CAM heatmaps, wherein, for each column, heatmap examples are provided for 8 thorax diseases which hold bounding boxes in official labeling, and in which the first row shows the results of the novel method, ACE, while the rest of the rows represent the localization of POPAR, DINO, BYOL and Adam, and the boxes are the localization ground truth.

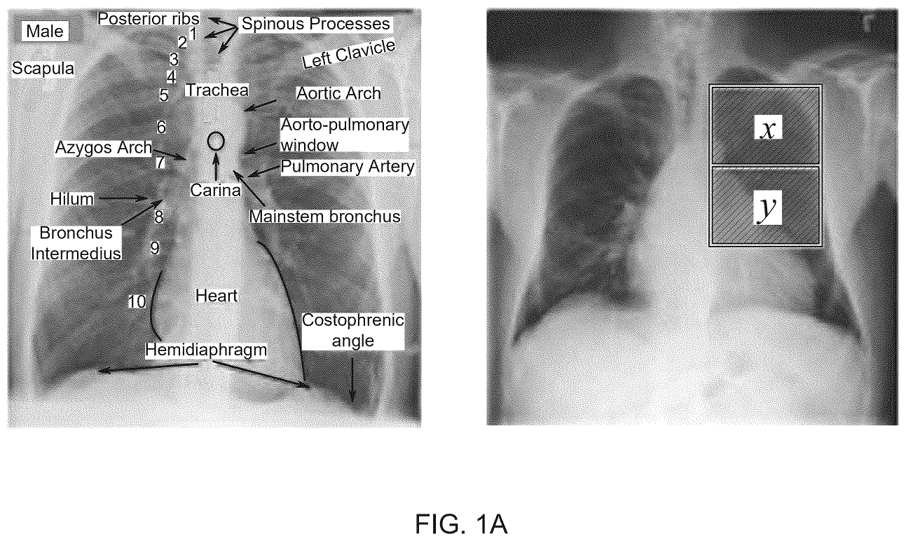

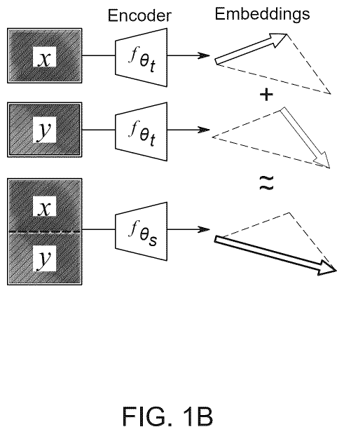

### DETAILED DESCRIPTION

Medical images acquired from standardized protocols show consistent macroscopic (global) or microscopic (local) anatomical structures, and these structures consist of composable/decomposable organs and tissues, but existing self-supervised learning (SSL) methods do not appreciate such composable/decomposable structure attributes inherent to medical images. To overcome this limitation, the disclosed embodiments introduce a novel SSL approach called ACE to learn Anatomically Consistent Embedding via composition and decomposition with two key branches: (1) a global consistency branch, capturing discriminative macro-structures via extracting global features; and (2) a local consistency branch, learning fine-grained anatomical details from composable/decomposable patch features via corresponding matrix matching. Experimental results across 6 datasets and 2 backbones, evaluated in few-shot learning, fine-tuning, and property analysis, show ACE's superior robustness, transferability, and clinical potential. Innovations of ACE include grid-wise image cropping, leveraging the intrinsic properties of compositionality and decompositionality of medical images, bridging the semantic gap from high-level pathologies to low-level tissue anomalies, and providing a new SSL method for medical imaging.

### I. INTRODUCTION

To test this hypothesis, the embodiments disclosed herein provide a framework that enables the capture of anatomical structures from unlabeled data, resulting in a powerful pretrained model. The embodiments are referred to herein as ACE because the embodiments learn Anatomically Consistent Embedding via an innovative composition and decomposition strategy, based on the idea that people parse visual scenes and model a viewpoint-invariant spatial relationship between part and whole. The framework consists of two novel components: learning global consistency and local consistency via composition and decomposition (detailed in Sec. 3). ACE utilizes novel grid-wise image cropping, which differs from the existing random cropping strategy, to provide truly precise patch matching. Based on this cropping strategy, two randomly cropped views input to a student-teacher model in the global consistency branch are guaranteed to have overlaps, reducing the feature irrelevances. In the local consistency branch, a student-teacher model is used to mimic the human understanding of part-whole relationships in images, where the embedding of a “whole” patch in one branch should always be consistent with the aggregated embedding of all “part” patches from the other branch, a process that is denoted herein as composition and decomposition. This process is based on precise patch matching, which differs significantly from the existing approximate matching methods because they compute local consistency by narrowing the distance of semantically closest or spatially nearest features. ACE simultaneously optimizes a loss that integrates global and local consistencies to learn anatomically consistent embedding.

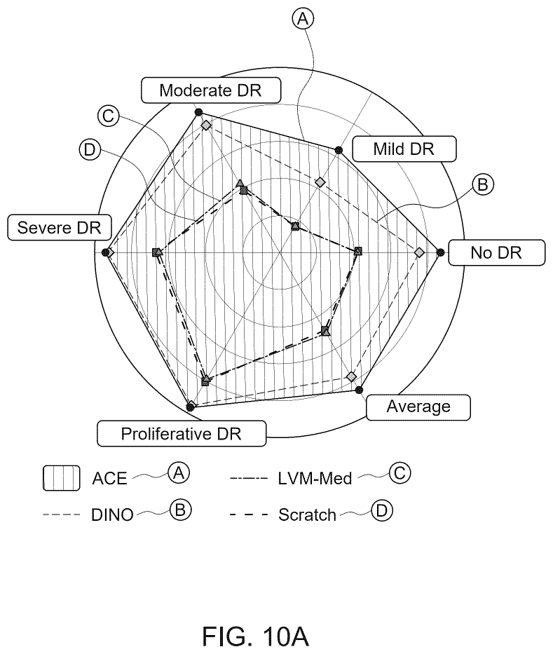

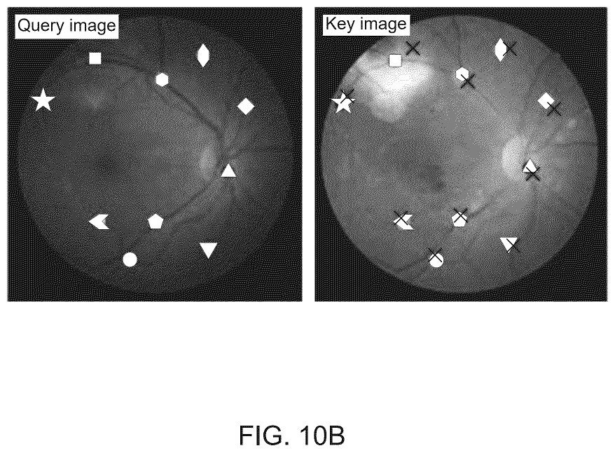

The disclosed embodiments as described herein focus on chest X-rays (CXRs), although it is appreciated that the disclosed embodiments are not limited to images of these particular anatomical aspects or structures of the human body. The disclosed embodiments were extensively evaluated in (1) exploring the learned and emergent properties: ACE is equipped with a set of unique properties by learning anatomies after pretraining (Sec. 5.1 and Sec. 5.2); (2) transferability to target tasks: ACE outperforms vision SSL methods designed for medical and photographic imaging and vision language SSL methods designed for medical imaging (Sec. 5.3); (3) generalization to other modality: ACE's adapting to fundus photography represents the universality to images acquired from standardized imaging protocols (FIG. 10B). The disclosed embodiments contribute:
a new target for learning compositionality and decompositionality from unlabeled medical images, demonstrating that deep models can comprehend anatomical structures in humans;
a novel exploration for pretrained backbone's properties including image retrieval, cross-patient anatomy correspondence, locality and symmetry of anatomical structures; and
a new SSL method with prominent transferability to various target tasks in medical image analysis.

### II. RELATED WORK

Self-supervised learning (SSL) methods share a common goal of learning meaningful representations without labeled data but differ in their primary learning focus: global features, local features, or structural patterns within images.

Learning global features and local features. A significant strand of SSL research concentrates on learning global features from images. These methods aim to capture the overall context of the image, ensuring consistency and alignment across different transformations of the input data. They can be grouped into two categories, contrastive learning and non-contrastive learning methods. This strand has progressively evolved to address challenges such as avoiding collapsing solutions and ensuring a balanced representation of global image features. However, these methods typically excel in capturing the consistency of macro-structures, but they often fail to capture the complexity of local details, compromising the accuracy needed for precise medical analysis. To address this, another research trajectory focuses on local feature learning whose objective is to distill fine-grained information by zooming in on specific parts of the image. This could involve detailed analysis of pixel-level features or segmenting images into smaller, coherent regions to learn representations that honor the local semantic content of the visual input. Current approaches align feature vectors of patches that are semantically closest or spatially nearest neighbors. However, in these ways, the local patch embeddings may not be precisely paired, which may confuse the model. The previous work, PEAC, focused on local anatomical consistency through precise local matching, but only considered positive local patches, ignoring unpaired ones. In contrast, the ACE approach described herein uses compositionality, decompositionality, and grid-wise patch matching to align matched local embeddings and separate mismatched ones. Through learning precise local consistency, the method according to the disclosed embodiments helps models capture fine-grained details, improving disease segmentation and classification in medical images.

Learning from structural patterns and anatomy. Another avenue within SSL is the exploration of structural patterns and anatomy. Medical imaging holds consistent anatomical structures, particularly when using the same protocol, and naturally provides supervision signals for models to learn anatomical representations through self-supervision. Previous studies have focused on reconstructing anatomical patterns from transformed images, understanding recurring anatomical patterns across different patients, exploring spatial relationships within anatomy, and enhancing these approaches with adversarial learning techniques. Although these methods focus on learning consistent representations of anatomy, they overlook the hierarchical relationships within anatomical structures. Additionally, the latest research, Adam-v2, exploits learning from composition and decomposition in a hierarchical way, but it remains limited to global patterns, overlooking the relationships in local patches. Unlike the existing methods, ACE learns from anatomy by utilizing the consistency of global patterns and compositionality alongside the decompositionality of local patterns, resulting in a hierarchical and integrative feature embedding structure.

### III. METHOD ACCORDING TO THE DISCLOSED EMBODIMENTS

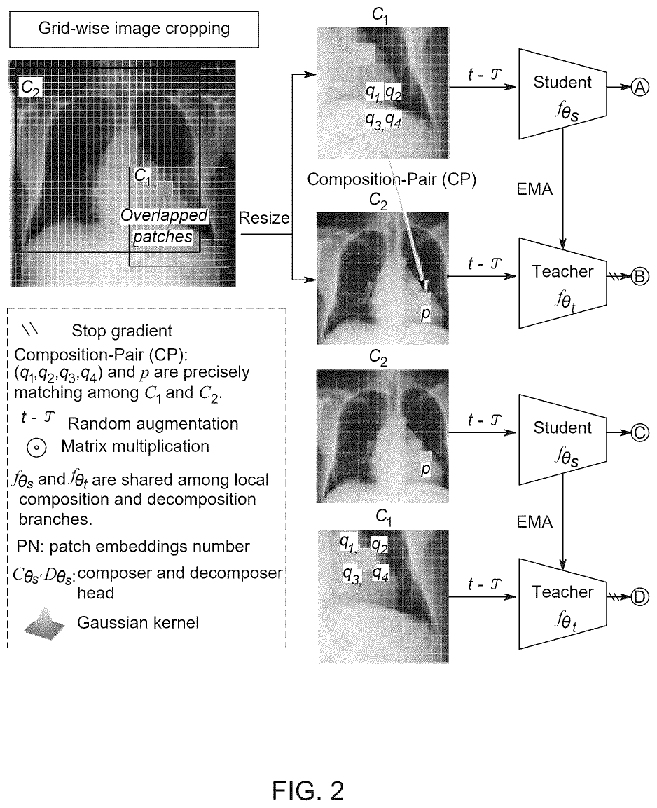

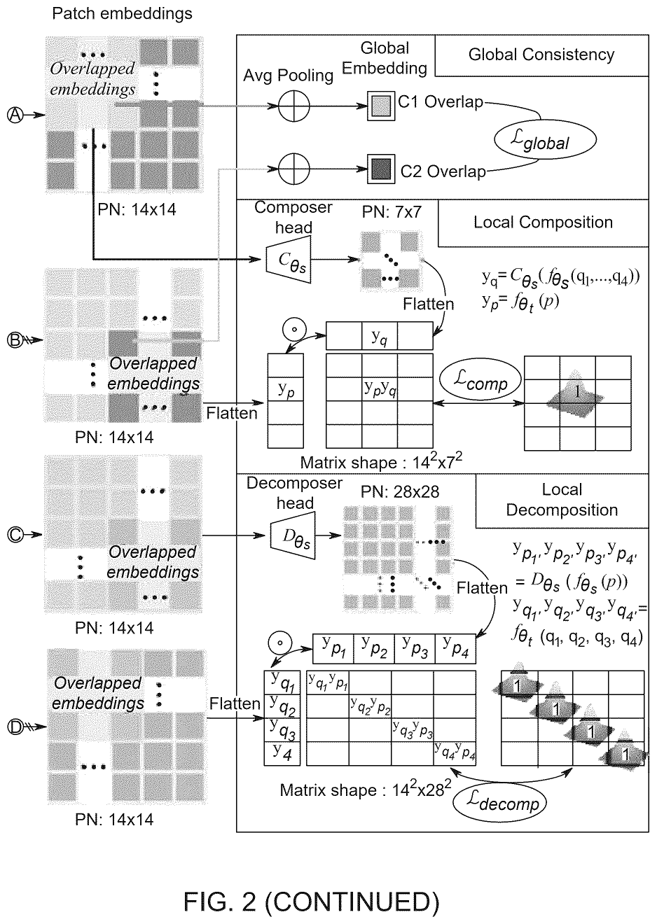

ACE, learning anatomies in global, local consistency via compositional and decompositional embeddings from unlabeled images, aims to bolster the development of self-supervised learning in medical imaging. The framework according to the disclosed embodiments is shown in FIG. 2 and comprises two parts: (1) a global consistency branch, which encourages the network to extract coarse-grained semantic features of different augmentations for the overlapped regions, and (2) a local consistency branch, which enforces the model to learn fine-grained local patterns via composition and decomposition. By integrating these components into a unified framework, ACE captures coarse to fine information in medical images, which provides powerful representations for various downstream tasks. The following discussion introduces methods from image pre-processing, each component to the joint training loss successively.

### 3.1 Image Pre-Processing: Grid-Wise Image Cropping

The embodiments of the disclosure introduce a grid-wise image cropping strategy to extract random image crops. First, the input image is divided into a grid G of 32×32 non-overlapping patches (see grids in FIG. 2), with each patch having a size m×m. Then, from the input image, two random crops are extracted (C₁ and C₂ in FIG. 2). C₁ comprises a 14×14 patch subset from G, and C₂ comprises a 28×28 patch subset from G. Based on the number of patches covered in each random crop of C₁ and C₂, in their overlap region, four patches in C₁, denoted as q₁, q₂, q₃, q₄, correspond to one patch in C₂, denoted as p={q₁, q₂, q₃, q₄}. In the reverse case, in the overlap region between C₁ and C₂, each patch (p) in C₂ corresponds to four patches (q₁, q₂, q₃, q₄) in C₁. In one embodiment of the disclosure, each patch has a size of 32×32 (i.e., m=32) and the input image is 1024×1024.

### 3.2 Learning Global Consistency

Global consistency motivates the network to extract consistent semantic features from various augmentations within the overlapping regions. After grid-wise image cropping, the two crops are resized to the same shape C₁, C₂∈R^{C×H}^{₀}^{×W}^{₀} C is the image channel and Hₒ, Wₒ represent the height and width. The resized crops C₁ and C₂ have different added augmentations x=T₁(C₁), x′=T₂(C₂) where T₁, T₂ are two transformations. Then the two crops are fed to the Student and Teacher models f_{θ}_{_{S}}, f_{θ}_{ₜ} to get patch embeddings yₛ, yₜ=f_{θ}(x), f_{θ}(x′)∈R^{K×N} respectively, where K is the embedding dimension,

$$N = \frac{{H}_{{oxW}_{o}}}{{m}^{2}}$$

and (m, m) is the resolution of each patch. Then the average pooling operator ⊕ is added to the overlapped patches to generate global embeddings y_{s⊕}=Avg(yₛ[O₁]), y_{t⊕}=Avg(yₜ[O₂])∈R^{K} where O₁ and O₂ are the overlapped areas of C₁ and C₂. The probability distributions P is obtained by normalizing the global embeddings with a softmax function:

$${P}_{S}^{( i )} = exp ( {y}_{⊕}^{( i )} / {T}_{s} ) / {\sum }_{k = 1}^{K} exp ( {y}_{s ⊕}^{( k )} / {T}_{s} )$$

where Tₛ>0 is a temperature parameter that controls the sharpness of the output distribution, and a similar formula holds for Pₜ with temperature Tₜ. Given a fixed teacher network f_{θ}_{ₜ}, embodiments learn to match these distributions by minimizing the cross-entropy loss:

$${ℒ}_{global} = {min}_{{θ}_{s}} CE ( {P}_{t} , {P}_{s} ) \qquad ( 1 )$$

where θs is the parameters of the student network, and CE(a, b)=−a log b.

### 3.3 Learning Local Consistency

Learning local consistency in composition. The local composition encourages the model to learn fine-grained anatomies in a part-to-whole manner by encouraging consistency from the integration of sub-patches to a bigger patch. For the overlapped patches in the two crops, the Composition-Pair (e.g., q₁, q₂, q₃, q₄ and p in FIG. 2) are precisely matching, and the composition is defined:

$$\text{?} = \text{?} ( \text{?} ( {q}_{1} , {q}_{2} , {q}_{3} , {q}_{4} ) ) \qquad ( 2 )$$

$$\text{?} \text{indicates text missing or illegible when filed}$$

where y_{q} is the compositional representation of the 4 sub-patches, C_{θ}_{ₛ} is composer head, and the bigger patch embedding yₚ=f_{θ}_{ₜ}(p), f_{θ}_{ₛ}, f_{θ}_{ₜ} are student and teacher encoders.

Embodiments learn local consistency by maximizing the similarity between paired patch embeddings and minimizing it for unpaired ones. A CLIP like cross-correlation matrix is used to guide the model in learning this consistency. In detail, when C₁ is input to student and C₂ to teacher model, yielding N embeddings for each crop: yₛ, yₜ∈R^{K×N}, and yₛ is input to composer head and it gets C_{θ}_{ₛ}(yₛ)∈R^{K×N/4} to merge each 2×2 embeddings into one embedding as Eq. 2. The cross-correlation matching matrix is defined as:

$${M}_{comp} = sigmoid ( {y}_{t}^{T} \cdot \text{?} ( {y}_{s} ) ) \qquad ( 3 )$$

$$\text{?} \text{indicates text missing or illegible when filed}$$

where in the matching matrix M_{comp}∈R^{N×N/4}, T is the transpose of a matrix, (⋅) is matrix multiplication, and the sigmoid function is added to restrict the values of the matrix to (0, 1). The value of the position (i, j),

$${M}_{c o m p}^{{( i , j )}_{=}} {{y}_{t} [ i ]}^{T} {C}_{{θ}_{s}} ( {y}_{s} ) [ j ]$$

represents the correlation between the two embeddings. Generally, the correlation weakens as the distance between their image patches increases. A Gaussian kernel G(x, y)=exp−(x²+y²)/(2σ²) is used to smooth the matching matrix target, assigning a value of 1 to exact matches and decreasing values as the distance increases. σ=1 and kernel size k=3 are set for implementation. The target matrix T_{comp}∈R^{N×N/4} whose value in the position (i, j):

$${T}_{comp}^{( i , j )} = \{ \begin{matrix}0 , if \vert Δ {x}^{( i , j )} \vert and \vert {Δ y}^{( i , j )} \vert > \frac{k - 1}{2} \\ exp ( - \frac{{\vert Δ {x}^{( i , j )} \vert}^{2} + {\vert {Δ y}^{( i , j )} \vert}^{2}}{2} ) , others\end{matrix} \qquad ( 4 )$$

where |Δx^{(i,j)}|, |Δy^{(i,j)}| are position distances between composed C₁ embedding C_{θ}_{ₛ}(yₛ)[j] and C₂ embedding yₜ^{[i]} after overlapped area alignment. The local composition learning loss:

$${ℒ}_{comp} = {min}_{{θ}_{s}} α CE ( {M}_{comp} , {T}_{comp} ) \qquad ( 5 )$$

where the hyper-parameter α=0.9 is used to balance the positive and negative samples.

Learning local consistency in decomposition. The local decomposition inspires the model learning consistency in a whole-to-part manner, which decomposes the patch embeddings into smaller sub-patches. As a symmetrical process of composition, decomposition learning lets inversely inputting: as shown in FIG. 2, C₂ to student and Cito teacher model to get yₛ, yₜ∈R^{KxN}. for a Composition-Pair, p is decomposed into 4 sub-embeddings:

$${y}_{{p}_{1}} , {y}_{{p}_{2}} , {y}_{{p}_{3}} , {y}_{{p}_{4}} = \text{?} ( {f}_{{θ}_{s}} ( p ) ) \qquad ( 6 )$$

$$\text{?} \text{indicates text missing or illegible when filed}$$

The smaller patch embeddings from teacher y_{q1}, y_{q2}, y_{q3}, y_{q4}=f_{θ}_{ₜ}(q₁, q₂, q₃, q₄), where f_{θ}_{ₛ}, f_{θ}_{ₜ}, D_{θ}, are student and teacher encoder, decomposer heads. yₛ is input to the decomposer head to decompose each 1 to 2×2 embeddings. The cross-correlation matching matrix for decomposition is

$${M}_{decomp} = sigmoid ( {y}_{𝔱}^{T} \cdot {D}_{{θ}_{s}} ( {y}_{s} ) ) ∈ {R}^{N \times 4 N} .$$

The decomposition matrix target T_{comp}∈R^{N×4N} whose value in position (i, j) is mathematically the same with Eq. 4. The local decomposition learning loss:

$${ℒ}_{decomp} = {min}_{{θ}_{s}} α CE ( {M}_{decomp} , {T}_{decomp} ) \qquad ( 7 )$$

where the hyper-parameter α=0.99 cis used to balance the positive and negative samples.

ACE according to the disclosed embodiments is illustrated in FIG. 2. ACE learns anatomically consistent embedding with two key branches: (1) a global consistency branch and (2) a local consistency branch via composition-decomposition. Using the grid-wise image cropping strategy (described herein in Sec. 3.1), an input image is divided into a grid (see grids in FIG. 2), and two random crops, C1 and C2, are extracted. In the overlap region between C1 and C2, four patches in C1 (denoted as q1, q2, q3, q4) correspond to one patch in C2 (denoted as p=q1, q2, q3, q4). The global consistency branch (described in Sec. 3.2) enforces consistency between the embeddings of the overlapping regions in C1 and C2 to learn coarse-grained semantic features. The local consistency branch (described in Sec. 3.3) enforces the model to learn fine-grained anatomical structure details via composition and decomposition. The local composition branch maximizes the similarity of paired patch embeddings and minimizes the similarity of unpaired ones to learn fine-grained anatomies in a part-to-whole manner. In a symmetrical process, the local decomposition branch enforces the model to learn fine-grained anatomies in a whole-to-part manner. An example pseudocode implementation of local consistency is provided below in Sec. 6.1 (A.1). The total loss is defined in Eq. 8, where L_{global} is the global loss empowering the model to learn coarse-grained anatomical structure from global patch embeddings. L_{comp}, L_{decomp} are two terms of local consistency loss equipping the model to learn precisely fine-grained local anatomical structures in composition and decomposition. λ1, λ2, λ3 are coefficients to balance the weights of each loss term.

$$ℒ = {λ}_{1} {ℒ}_{global} + {λ}_{2} {ℒ}_{comp} + {λ}_{3} {ℒ}_{decomp} \qquad ( 8 )$$

### IV. IMPLEMENTATION DETAILS

Pretraining settings. The composer and decomposer heads are 2-layer MLPs to integrate and expand the local embeddings. The coefficient values of total loss are λ₁=0.1, λ₂=λ₃=1. ACE is pretrained on unlabeled ChestX-ray14 dataset with Swin-B and ViT-B backbones with 448² image sizes training for 100 epochs. ACE was compared with and evaluated against a variety of SSL methods developed for ResNet, Vision Transformer and Swin-Transformer architectures. These methods respectively leverage global information: DINO, BYOL; patch-level information: SelfPatch; and the structural patterns: Adam, POPAR, and DropPos. For equal comparison, the same experimental settings are used with ACE and these methods pretrained with ACE and with ChestX-ray14 dataset. ACE is also compared with vision-language model KAD, ChexZero and DeViDe which are pretrained on chest X-ray images MIMIC-CXR dataset, and the pretrained models directly loaded for downstream comparisons. More details are described below in Sec. 6.1 (A.3).

Fine-tuning settings. Pretrained models are fine tuned in supervised settings on downstream tasks including classification and segmentation. Classification performance is validated on 3 thoracic disease classification tasks ChestX-ray14, Shenzhen CXR, RSNA Pneumonia. For the segmentation task, the dense prediction performance is validated on JSRT, ChestX-Det and SIIM. The pretrained models are transferred to each target task by fine-tuning the whole parameters. The AUC (area under the ROC curve) metric is utilized to assess the performance of multi-label classification tasks on datasets such as ChestX-ray14 and Shenzhen CXR and for RSNA Pneumonia, accuracy is used as the evaluative measure. For the target segmentation task, UperNet is used as the training model and adds an additional randomly initialized prediction head. The Dice is used to evaluate the segmentation performance. More detailed settings including hyper-parameters are described in Sec. 6.1 (A.2), and Sec. 6.1 (A.4), below.

### V. RESULTS

This section highlights the core results of the study, demonstrating the significance of the self-supervised learning framework, ACE, according to the disclosed embodiments. First, the properties are introduced on which ACE was explicitly trained to learn (Sec. 5.1). Next, the emergent properties of ACE are revealed that were not part of its explicit training (Sec. 5.2). Finally, extensive experiments were conducted to showcase ACE's generality and adaptability across various tasks (Sec. 5.3), evaluated through two key aspects: (i) data efficiency, and (ii) fine-tuning settings.

### 5.1 Learned Properties

### (1) ACE Enhances Feature Compositionality.

Experimental Setup: ACE's ability to preserve the compositionality of anatomical structures in its learned embedding space is investigated. Patches are randomly extracted from test images in the ChestX-ray14 dataset and each patch is further decomposed into 2 or 4 non-overlapping sub-patches. Each extracted patch and its sub-patches are then resized to a fixed dimension (i.e., 448×448) and their features are extracted using ACE's pre-trained model as well as other baseline pretrained models. Subsequently, the cosine similarity between the embedding of each patch and the average embedding of its sub-patches is calculated, and the similarity distributions are visualized using Gaussian kernel density estimation (KDE).

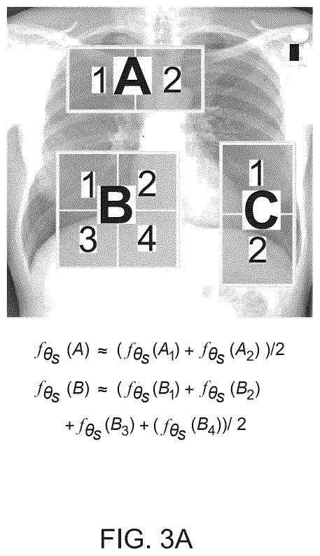

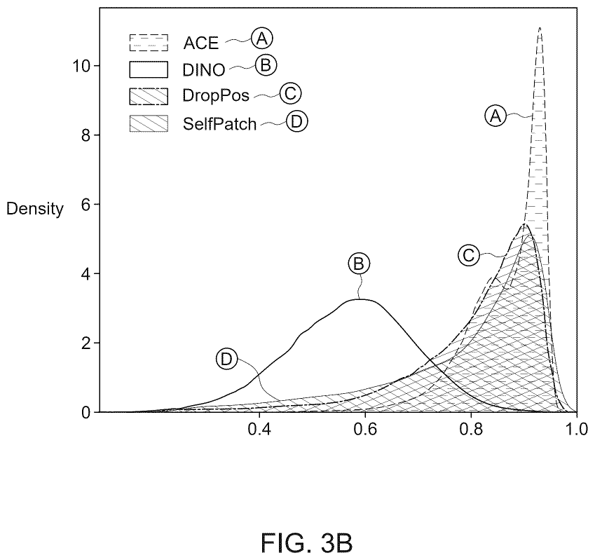

Results: As shown in FIGS. 3A and 3B, ACE's distribution not only exhibits a narrower and taller shape compared with the baselines, but also has the mean similarity value between the embeddings of patches and their compositional parts (sub-patches) shifted towards 1. These observations demonstrate ACE's effective integration of cohesive features while maintaining the compositional integrity of anatomical structures, echoing its ability to preserve the compositionality of anatomical structures in its learned embeddings.

### (2) ACE Enhances Feature Decompositionality.

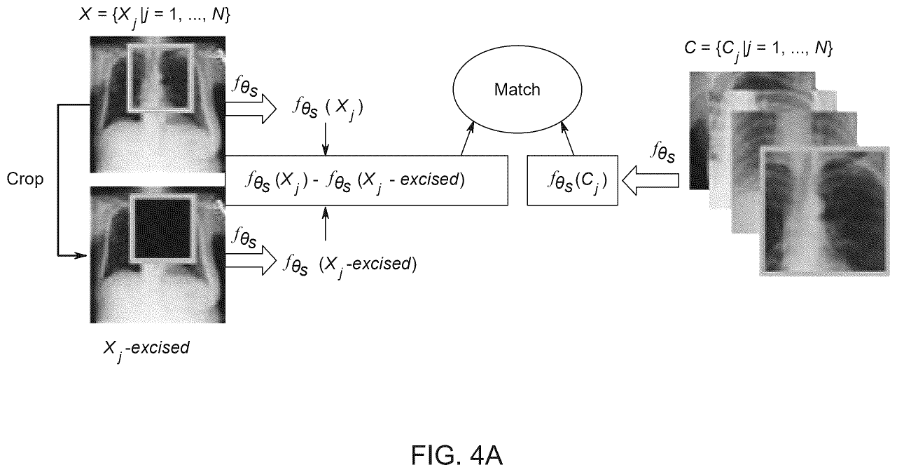

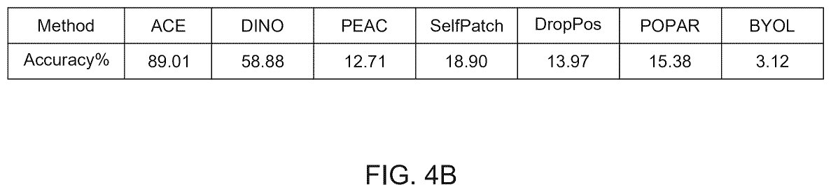

Experimental Setup: ACE's ability to maintain the decompositionality of anatomical structures in its learned embedding space is examined. To do so, the test set of the ChestX-ray14 dataset is first divided into batches, each containing 32 images. In each batch, a random patch is extracted (from each image), labeled as Cⱼ in FIG. 4A, sized between 30-60% of the original image. The original image is then passed, the image with the region is removed (labeled as X_{j-excised}), and the extracted patches (excised region Cⱼ) are passed to the ACE's pretrained model and other baseline pretrained models to extract their features. Finally, the cosine similarity between f_{θ}s (Xⱼ)−f_{θ}s (X_{j-excised}) and f_{θ}s (Cⱼ) is calculated, verifying if f_{θ}s (Xⱼ)−f_{θ}s (X_{j-excised})≈f_{θ}s (Cⱼ).

Results: As seen in FIG. 4B, ACE surpasses the SSL baselines by a remarkable margin. Notably, compared with DINO, PEAC, SelfPatch, DropPos, POPAR, and BYOL, which achieve an accuracy of 58.88%, 12.71%, 18.90%, 13.97%, 15.38%, and 3.12% respectively, ACE achieves a high accuracy of 89.01%. This substantial difference in accuracy highlights ACE's superior ability to preserve the decompositionality of anatomical structures in its learned embeddings.

### (3) ACE Provides Robust Local Feature-Driven Global Image Retrieval.

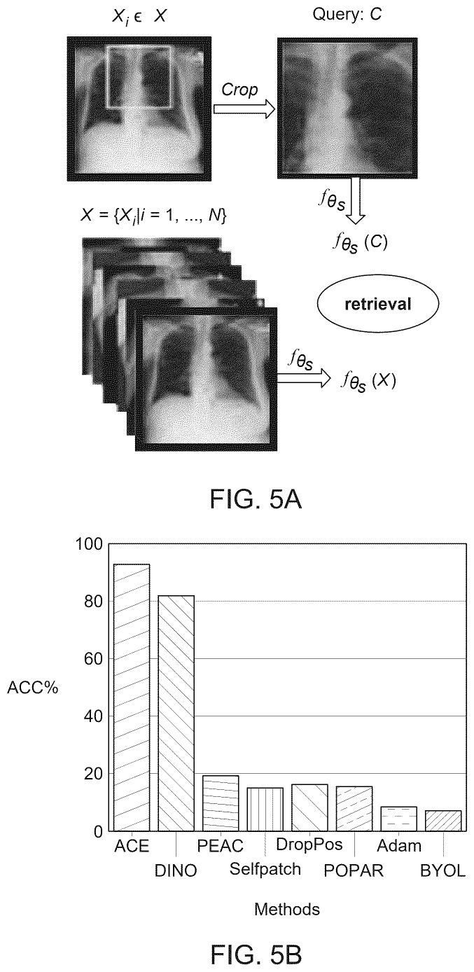

Experimental Setup: ACE's ability to capture semantics-rich features in its learned embedding space is explored. To do so, the test set of the ChestX-ray 14 dataset is divided into batches, each comprising 32 images. For each batch, a random image X is selected and a random patch extracted from it to use as the query, denoted as C in FIG. 5A. Using ACE's pretrained model and other baseline models, features were extracted for the query patch (f_{θ}s (C)) as well as for each whole image in the batch f_{θ}s (Xᵢ)|Xᵢ∈X. The cosine similarity between the query patch's embedding and the embeddings of the whole images in the batch were then calculated. A retrieval is considered correct if the highest cosine similarity score corresponds to the query patch and its associated whole image in the batch.

Results: As seen in FIG. 5B, ACE achieves the highest retrieval accuracy (94.37%) compared with other SSL baselines, demonstrating the semantic richness of ACE's learned representations. This result highlights ACE's clinical potential for accurately identifying and retrieving patients with similar pathological findings based on a query patch related to a specific disease.

### 5.2 Emergent Properties

The following properties are considered “emergent” as ACE is never trained with global and local consistencies across patients but such inter-image consistency has automatically emerged from training on intra-image consistency.

### (1) ACE Provides Distinctive Anatomical Embeddings.

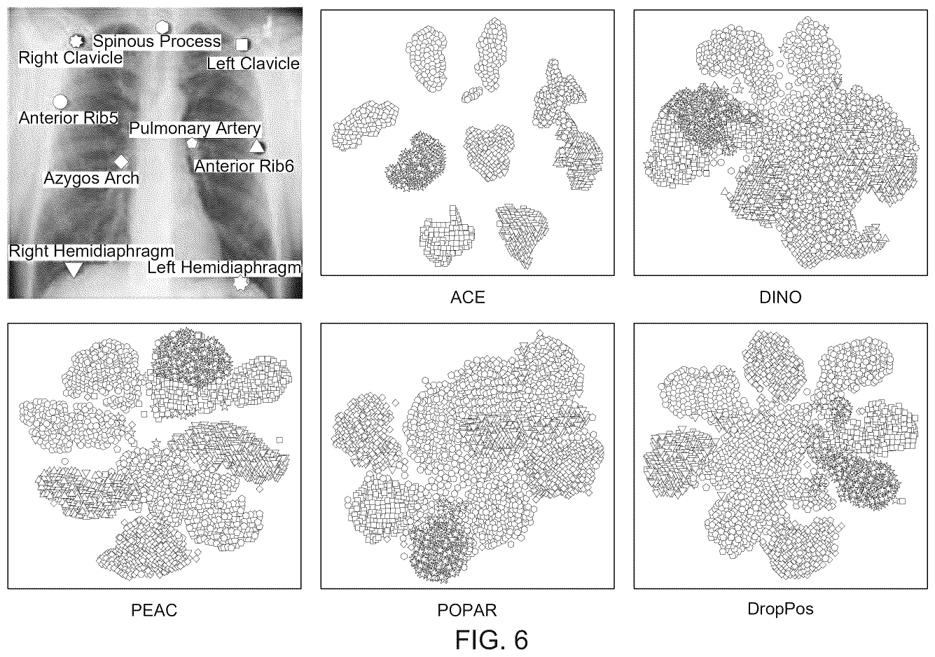

Experimental Setup: ACE's ability to reflect the locality of anatomical structures in its learned embedding space is investigated. To do so, a dataset of 1,000 images is compiled from the ChestX-ray14 dataset, each annotated by experts with 9 distinct anatomical landmarks (see FIG. 6). 448² patches are extracted around each landmark's location from the 1024² original images and then latent features extracted for each landmark instance using ACE's pretrained model and other baseline pretrained models (without fine-tuning). These features are visualized using a t-SNE plot.

Results: As seen in FIG. 6, the SSL baselines—DINO, PEAC, POPAR and DropPos—struggle to generate distinct features for different landmarks, leading to ambiguous embedding spaces with mixed clusters. However, ACE excels at distinguishing between various anatomical landmarks, resulting in well-separated clusters within its learned embedding space. This emphasizes ACE's capability to develop a rich embedding space, where different anatomical structures are uniquely represented, and identical anatomical structures across patients have closely similar embeddings.

### (2) ACE Provides Unsupervised Cross-Patient Anatomy Correspondence.

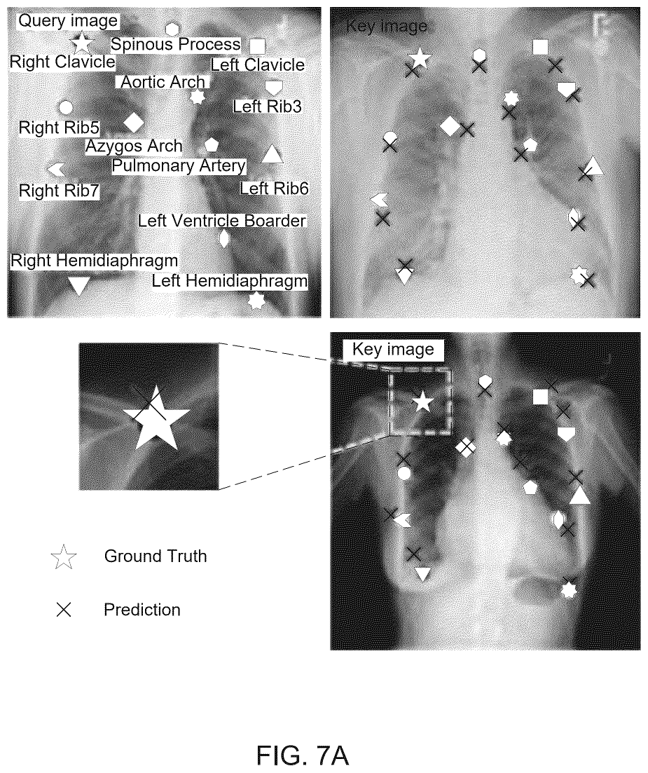

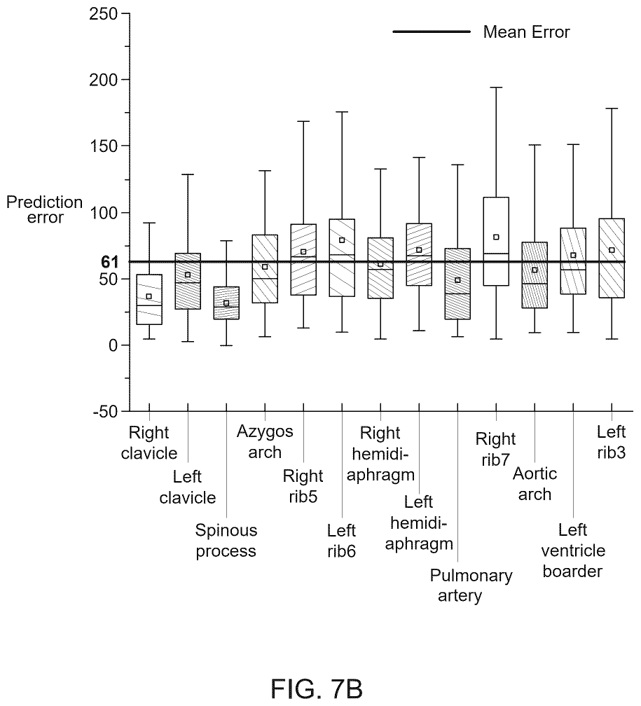

Experimental Setup: To demonstrate the efficacy of ACE in capturing a diverse range of anatomical structures, patch-level features are used to query the same anatomy across different patients in a zero-shot setting. In detail, the dataset mentioned in Sec. 5.2 is used and N_{q}=13 landmarks are chosen and labeled by human experts shown in FIG. 7A. For a given query image, patches of size 448² centered at each landmark point will be extracted from the initial size 1024² image. These patches will be input to ACE's pre-trained backbone (no fine-tuning) to get the query features of the centered landmarks

$${E}_{q} = \{ {E}_{j}^{i} \} \begin{matrix}{N}_{q} \\ i = 1.\end{matrix}$$

Then for the rest of the key images, Nₖ patches are extracted by sliding a window of size 448² with a stride of 8 (zero padding for the boundary patches), then these patches are input to ACE backbone to get a dictionary of features for the key image

$${E}_{k} = \{ {E}_{k}^{j} \} \begin{matrix}{N}_{k} \\ j = 1.\end{matrix}$$

Finally, for each query landmark feature in E_{q}, feature in Eₖ with l₂ distance is found and the position in the key image is the prediction corresponding landmark.

Results: The predicting and ground truth landmarks are plotted in FIG. 7A, and the prediction errors analyzed with a box plot of each landmark shown in FIG. 7B. From the results, the anatomical landmarks can be precisely detected using ACE encoded features and the average error of 13 landmarks is 61 pixels in 1 k size of 1024² images. These findings indicate that the extracted features reliably represent specific anatomical regions and maintain consistency despite significant morphological variations.

### 5.3 Downstream Transferability of ACE

### (1) Data Efficiency Evaluation.

Experimental Setup: Robustness of ACE's representations in limited data regimes is dissected. To do so, a pretrained ACE model is compared with two SSL pretrained models, POPAR and DINO, by fine-tuning the models with a few labeled data (2, 5 and 10 shots) from the JSRT-Heart dataset and with limited labeled fractions (1%, 10% and 50%) from the SIIM dataset.

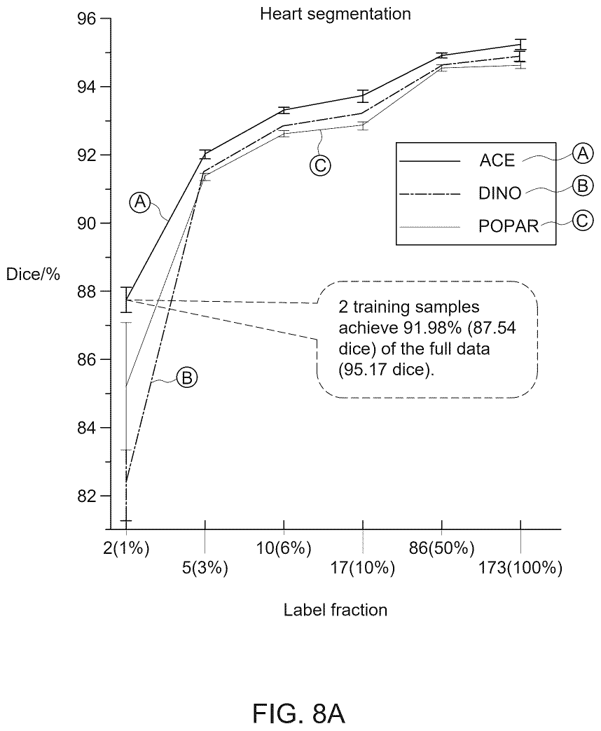

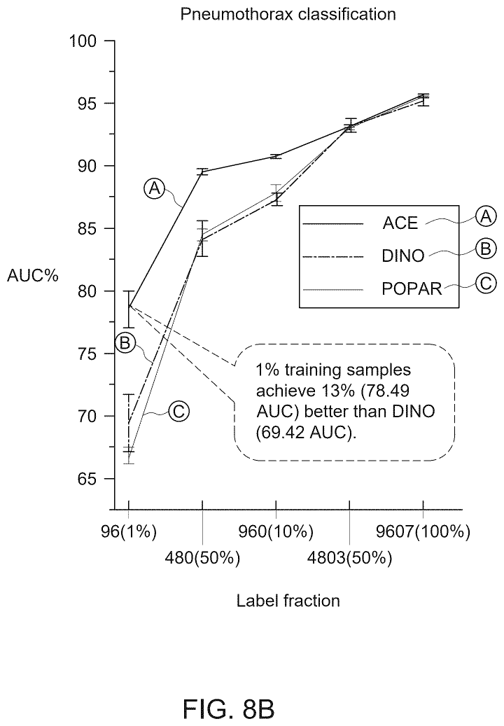

Results: As seen in FIGS. 8A and 8B, ACE outperforms POPAR and DINO models in limited data regimes for both heart segmentation and pneumothorax classification tasks. Notably, in the heart segmentation task, ACE achieves over 91% of its full training data performance using only 2 labeled samples. These results highlight ACE's annotation efficiency, particularly in target tasks where labeled data is scarce.

### (2) Fine-Tuning Evaluation.

Experimental setup: The generalizability of ACE's representations in full fine-tuning settings across a wide range of downstream tasks is investigated. To do so, ACE is compared with 9 SSL baselines with diverse objectives in 3 classification tasks and 4 segmentation tasks. Also, the performance of training from scratch is included as a lower bound.

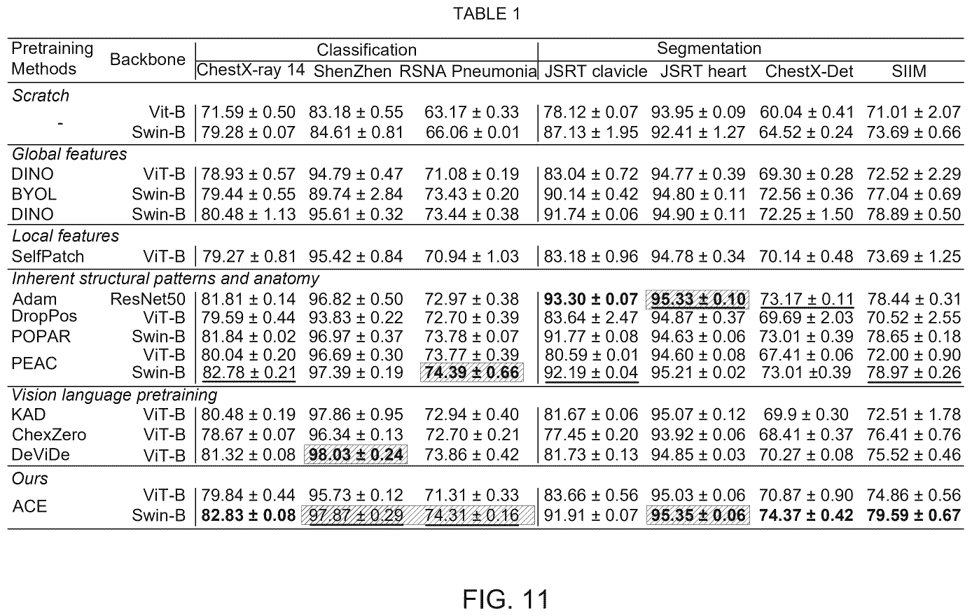

Results: As seen in Table 1 presented in FIG. 11, ACE model with the ViT-B backbone delivers competitive or superior performance compared with baselines with the same backbone, including DINO, SelfPatch, and DropPos. Moreover, ACE with the Swin-B backbone consistently outperforms BYOL, DINO, and POPAR, all of which also use the Swin-B backbone, across all downstream tasks. Comparing with methods adapted to medical imaging, including the vision SSL models POPAR, Adam and vision language SSL methods KAD, ChexZero, DeViDe, ACE can give the best or the next best performance. These results demonstrate the higher transferability and generalizability of ACE's representations across various tasks.

### VI. ABLATION STUDY

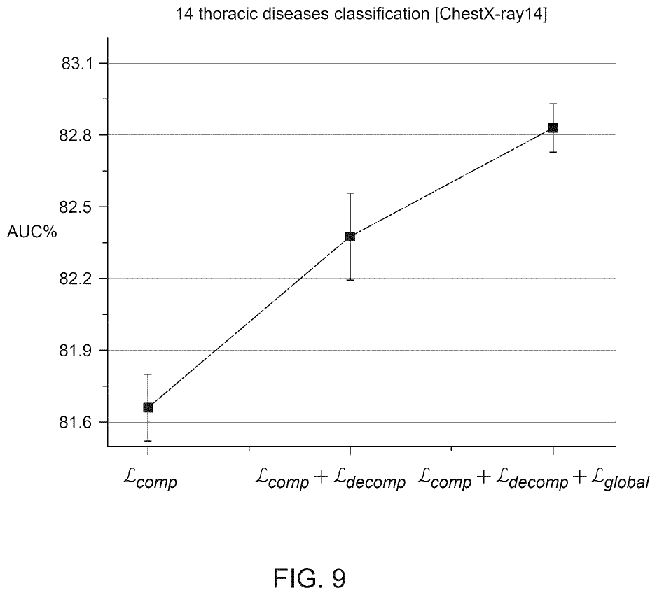

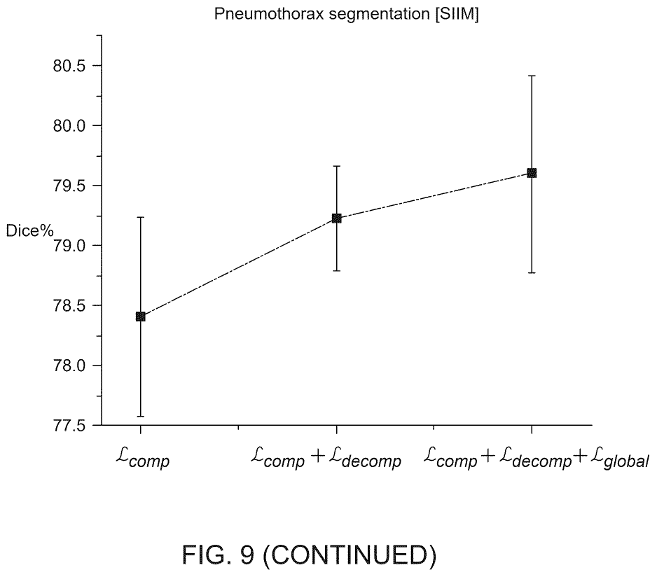

Effectiveness of the learning Objects. The impact of each learning component in ACE is evaluated by progressively incorporating decompositionality and global loss, starting with compositionality. The models are fine-tuned on two tasks: the ChestX-ray14 dataset for thoracic disease classification and the SIIM dataset for pneumothorax segmentation. As shown in FIG. 9, performance improves across both tasks with the addition of each loss component, highlighting the effectiveness of each.

Generalizability of ACE. A framework according to the disclosed embodiments can be seamlessly extended to other imaging modalities. To demonstrate this, ACE is pretrained on unlabeled fundus images EyePACS and fine-tuned on diabetic retinopathy classification dataset EyePACS. As seen in FIG. 10A, ACE exhibits superior performance compared with SOTA SSL method DINO, large-scale pretraining method LVM-Med and training from scratch. Besides, unsupervised anatomy correspondence was conducted based on ACE's pre-trained backbone without fine-tuning on fundus image registration dataset FIRE. As shown in FIG. 10B, the key points corresponding with the query image can be precisely located in the key image.

### 6.1 Additional Material for ACE: Anatomically Consistent Embeddings in Composition and Decomposition

### A. Implementation Details

### A.1. Pseudo Code Implementation

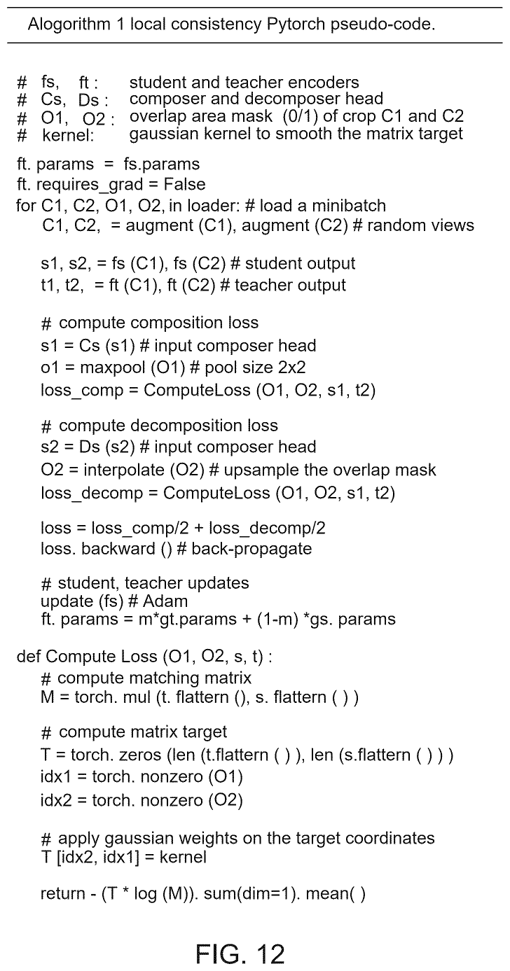

A pseudo-code implementation of local consistency is proposed below in Algorithm 1 presented in FIG. 12, in accordance with the disclosed embodiments.

### A.2. Pretraining and Testing Datasets

ACE is evaluated on chest X-rays and fundus photography, and pretrained on ChestX-ray14 and Eye-PACS datasets respectively. The pretrained ACE models are validated on target tasks including the following datasets:

ChestX-ray14, which contains 112K frontal view X-ray images of 30805 unique patients with the text-mined fourteen disease image labels (where each image can have multi-labels). The official training set 86K (90% for training and 10% for validation) and testing set 25K is used. The downstream models are trained to predict 14 pathologies in a multi-label classification setting and the mean AUC score is utilized to evaluate the classification performance. In addition to image-level labeling, the datasets provide bounding box annotations for 880 images in a test set. Of this set of images, bounding box annotations are available for 8 out of 14 thorax diseases. After fine tuning, bounding box annotations are used in a test set to assess the accuracy of pathology localization in a weakly supervised setting. Besides, a dataset of 1,000 images is compiled from a test set, each annotated by experts with distinct anatomical landmarks. These labeled landmarks are used for anatomical embeddings analysis (see discussion above, Sec. 5.2 (1) and below in Sec. 6.1 (B.1), unsupervised key-point correspondence (see discussion above, Sec. 5.2 (2), key-point detection (see Sec. 6.1 (B.2).

NIH Shenzhen CXR, which contains 326 normal and 336 Tuberculosis (TB) frontal-view chest X-ray images, split 70% of the dataset for training, 10% for validation and 20% for testing.

RSNA Pneumonia, which consists of 26.7K frontal view chest X-ray images and each image is labeled with a distinct diagnosis, such as Normal, Lung Opacity and Not Normal (other diseases). 80% of the images are used to train, 10% to validate and 10% to test.

JSRT, which is an organ segmentation dataset including 247 frontal view chest X-ray images. All images are in 2048×2048 resolution with 12-bit grayscale levels. The heart and clavicle segmentation masks are utilized for this dataset. 173 images are split for training, 25 for validation and 49 for testing.

ChestX-Det, which is a disease segmentation dataset and an improved version of Chest-XDet10. This dataset contains 3,578 images with instance-level annotations for 13 common thoracic pathology categories, sourced from the NIH ChestX-ray14 dataset. Annotations were provided by three board-certified radiologists, and the dataset includes additional segmentation annotations. All the diseases were consolidated into one region and the goal of segmenting this dataset is to distinguish between diseased and non-diseased areas for each image. There is an official split for training and testing sets and a split of 10% of images from the training set for validation.

SIIM-ACR, a dataset resulting from a collaboration between SIIM, ACR, STR, and MD.ai, contains 12,089 chest X-ray images. It is the largest public pneumothorax segmentation dataset to date, comprising 3,576 pneumothorax images and 9,420 non-pneumothorax images, all of which are available in 1024×1024 pixel resolution. The dataset is randomly divided into training (80%), validation (10%) and testing (10%). The segmentation performance is measured by the mean Dice which averages the dice of pneumothorax non-pneumothorax images.

EyePACS, a diabetic retinopathy (DR) classification dataset for identifying signs of diabetic retinopathy in eye images. The clinician has rated the presence of diabetic retinopathy in each image on a scale of 0 to 4, 0 for no DR, 1 for mild DR, 2 for moderate DR, 3 for severe DR and 4 for proliferative DR. There are 53,576 unlabeled images and 35,126 with labels. The labeled images are randomly split into training (80%), validation (10%) and testing (10%) sets for downstream evaluation, and the training, validation and unlabeled sets are merged for pretraining.

FIRE, the dataset comprises 134 pairs of images obtained from 39 patients, with each pair annotated with specific corresponding key points. In target tasks, for each pair of images, one image is designated as the query image, and the task is to identify the corresponding anatomical structures in the key image. Additionally, the predicted and ground truth key points in the key image are simultaneously visualized.

### A.3. Pretraining Settings

There are two trained ACE models with Swin-B backbone using unlabeled images from ChestX-ray14 and Eye-PACS for the adaptation on chest X-ray and fundus imaging. Moreover, to generalize to other architecture ACE was trained on ViT-B backbone on ChestX-ray14. The ACE learning paradigm is similar to knowledge distillation [7], where a student network learns to match a teacher network's output. The weights of the student model θₛ are updated by back-propagation and the gradients of the teacher model are stopped whose weights θₜ are updated using EMA (exponential moving average) from the student model. The update rule is θₜ←λθₜ+(1−λ)θₛ, where λ follows a cosine schedule from 0.996 to 1 during training.

The composer and decomposer heads are 2-layer MLPs to integrate and expand the local embeddings. In detail, the output of student or teacher encoder are patch embeddings with shape 14×14×1024. Before the composer head, each 2×2×1024 adjacent embeddings are concatenated and the patch embeddings are reshaped to 7×7×4096, then they are input to a 2-layer MLP with input dimension 4096 and output dimension 1024 to get a shape of 7×7×1024 embeddings. Symmetrically, in the decomposer head, the 14×14×1024 patch embeddings are input to a 2-layer MLP with input dimension 1024 and output dimension 4096 to expand the embeddings to 14×14×4096, then each embedding is chunked into 2×2×1024 and the output embeddings will be 28×28×1024.

During the pretraining phase, a batch size of 8 images per GPU is used and trained for a total of 100 epochs with 4 V100 (32G). The optimizer is AdamW and the initial learning rate is set to 5e-4 with a linear warm-up over the first 10 epochs. The weight decay starts at 0.04 and reaches 0.4 by the end of training, following a cosine schedule. The drop path rate is set to 0.1. Gradient clipping is applied with a maximum norm of 0.8 to ensure stable training dynamics.

### A.4. Fine Tuning Settings

For the target classification tasks, a randomly initialized linear layer is concatenated to the output of the classification (CLS) token of ViT-B pretrained models. For Swin-B pretrained models, an average pooling is added to the last layer feature maps, then the feature fed to the randomly initialized linear layer. For the target segmentation task, UperNet is used as the training model. Pretrained weights are concatenated with a randomly initialized prediction head for segmenting. The AdamW optimizer is used in conjunction with a cosine learning rate scheduler. A linear warm-up phase spanning 20 epochs is used, within a total training duration of 150 epochs. The base learning rate is set at 0.0001. Each experiment is conducted using four V100 32 GPUs, with a batch size of 32 per GPU. For segmentation tasks, the same setup is retained and the training period extended to 500 epochs.

### B. Additional Results

### B.1. Emergent Property: ACE Understand Anatomical Symmetry

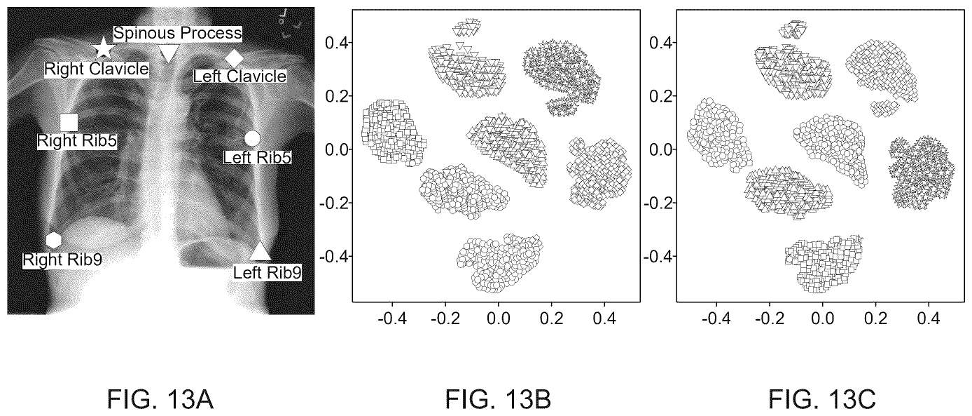

Experimental Setup: ACE's ability to capture the symmetry of anatomical structures in its learned embedding space is examined. To do so, N=7 anatomical landmarks are considered, including three pairs of mirrored structures and one structure located in the center of the chest, as shown in FIG. 13A. A size of 448² patches

$$( C = \{ {C}_{i} \} \begin{matrix}N \\ i = 1\end{matrix} )$$

is extracted around each landmark's location from the original images, and then ACE's pretrained model is used to extract latent features for each landmark and its corresponding left and right flipped version (Č=T (C)). The extracted features of C and Č are visualized via t-SNE plots in FIGS. 13B and 13C, respectively.

Results: As seen in FIGS. 13B and 13C, ACE captures the symmetry of anatomical structures within its learned embedding space. For example, the right and left clavicles, which are visually symmetrical, are represented similarly in the embedding space. As seen, the cluster in FIG. 13B, corresponding to the right clavicle, closely matches the cluster in FIG. 13C, which represents the flipped left clavicle. A similar pattern is observed for other pairs, such as the left rib 5 and its flipped version, represented by the clusters in FIGS. 13B and 13C, respectively. These observations demonstrate that ACE effectively captures the symmetry of anatomical structures in its learned embedding space as an emergent property.

### B.2. Fine-Tuning Evaluation: Key Point Detection.

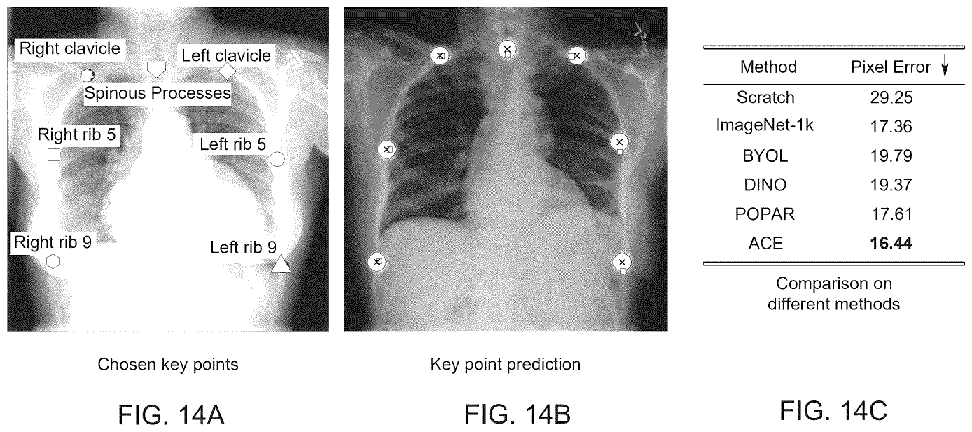

Experimental Setup: The generalizability of ACE's pretrained model is investigated via fine-tuning the landmark detection task. To do so, the dataset annotated by experts with distinct anatomical landmarks (mentioned above in Sec. 5.2) is used, and 7 key points were chosen as shown in FIG. 14A. The pretrained weights of ACE and other baselines including ImageNet-1K, BYOL, DINO and POPAR were loaded. The fine-tuning architecture is UperNet which is the same with segmentation, while the training target is the specific points of interest. The detection process is optimized based on the heatmap method, that is, a 11×11 Gaussian kernel exp

$$( - \frac{{x}^{2} + {y}^{2}}{{2}_{{σ}^{2}}} ) ,$$

which is added to smooth each ground truth landmark where the peak is 1 and the values decrease as the distance increase. The learning target is visualized in FIG. 14B where the crosses are the center of the heatmaps. The error between prediction and ground truth points is used as the evaluation metric.

Results: As seen in FIG. 14C, initializing with ACE's weights can get the lowest pixel error 16.44 while the image size is 448×448, better than initialized with other baselines, ImageNet pretrained weights and training from scratch. From the results, ACE's representations can give some priors about the anatomical structure which boosts to distinguish the key points.

### B.3. Weakly Supervised Localization.

Experimental Setup: To compare with other pretraining methods POPAR, DINO, BYOL and Adam, the downstream model was initialized with these pretrained weights using only image-level disease label on ChestX-ray14 dataset. After fine-tuning, the models are used for inference on 787 cases annotated with bounding boxes for eight thorax diseases: Atelectasis, Cardiomegaly, Effusion, Infiltrate, Mass, Nodule, Pneumonia, and Pneumothorax. A Grad-CAM heatmap was used to approximate the localization of a specific thorax disease predicted by the trained model. The baseline Adam is fine-tuned on ResNet50 and other methods are based on Swin-B.

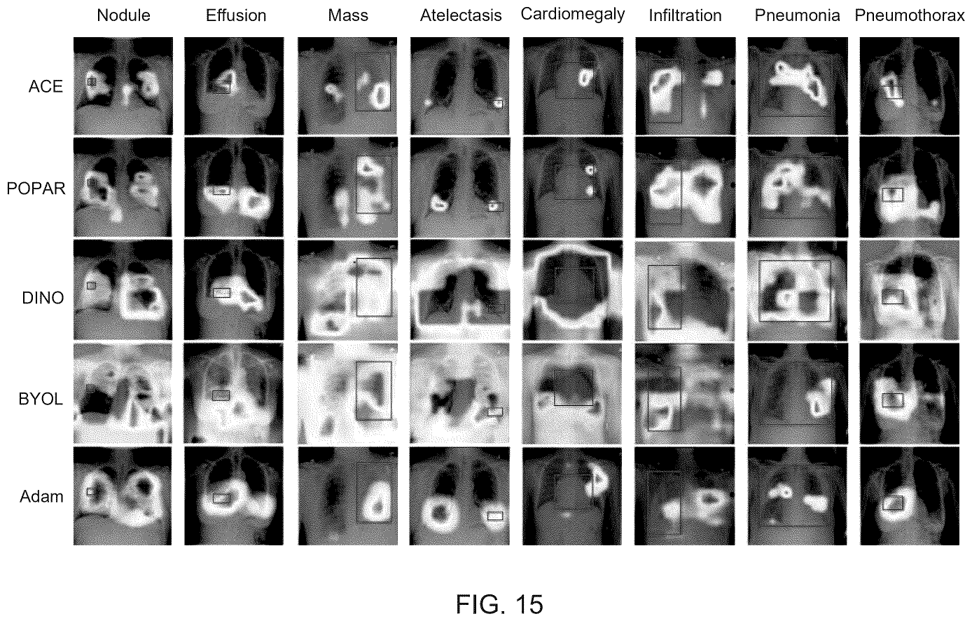

Results: FIG. 15 shows the visualization of heatmaps generated by ACE, POPAR, DINO, BYOL and Adam for 8 thorax pathologies in ChestX-ray14 dataset. From the results, the localization of the method according to the disclosed embodiments surpasses the learning global feature methods DINO and BYOL and learning inherent structure pattern method POPAR and Adam. For analyzing the Grad-CAM heatmaps, the disclosed method shows more precise and compact localization with small shifts, while the learning global feature methods DINO and BYOL often completely cannot localize the diseases. And surprisingly, the novel model can also localize some small pathologies like nodules and atelectasis, which demonstrate the positive impact of the combination of learning global and local anatomies.

### VII. CONCLUSION

Thus, the embodiments of the disclosure provide a novel SSL method aimed at visual representation learning via composition and decomposition for anatomical structures in medical images. The novel method relies on learning global consistency and local consistency by reliable global representation alignment and correspondence matrix matching. The embodiments have been rigorously tested through comprehensive experiments in various tasks, demonstrating emergent properties and effective transferability, showing significant promise for advancing and explainable AI applications in medical image analysis.

What is claimed is:

1. A computer-implemented method for a self-supervised learning (SSL) model to learn an anatomically consistent embedding from unlabeled medical images, comprising:
receiving a plurality of unlabeled medical images including consistent global and local anatomical structures;
dividing each of the plurality of unlabeled medical images into a grid of non-overlapping patches;
extracting two random crops from each of the plurality of unlabeled medical images, wherein each of the two random crops comprises a subset of the grid of non-overlapping patches and shares with the other of the two random crops a partially overlapping region of the grid of non-overlapping patches;
executing a global consistency branch that captures discriminative macro-structures in the plurality of unlabeled medical images by extracting global embeddings; and
executing concurrently a local consistency branch that learns fine-grained local anatomical details in the plurality of unlabeled medical images via composition and decomposition operations.

2. The computer-implemented method of claim 1 wherein executing the global consistency branch that captures discriminative macro-structures in the plurality of unlabeled medical images by extracting global embeddings, comprises extracting coarse-grained semantic features of different augmentations within the overlapping regions of each of the plurality of unlabeled medical images.

3. The computer-implemented method of claim 2, wherein extracting coarse-grained semantic features of different augmentations within the overlapping regions of each of the plurality of unlabeled medical images comprises:
resizing the two random crops to a same shape;
adding different augmentations to the resized crops;
feeding the augmented resized crops to a student model and a teacher model to obtain overlapping patch embeddings;
adding an average pooling operator to the overlapping patch embeddings to obtain global embeddings; and
obtaining a plurality of probability distributions by normalizing the global embeddings with a softmax function.

4. The computer-implemented method of claim 1, wherein executing concurrently the local consistency branch that learns fine-grained local anatomical details in the plurality of unlabeled medical images via composition and decomposition operations, comprises integrating the subset of the grid of non-overlapping patches that each of the two random crops from each of the plurality of unlabeled medical images shares with the other of the two random crops into a single, larger, patch.

5. The computer-implemented method of claim 4, wherein executing concurrently a local consistency branch that learns fine-grained local anatomical details in the plurality of unlabeled medical images via composition and decomposition operations further comprises integrating the subset of the grid of non-overlapping patches that each of the two random crops from each of the plurality of unlabeled medical images shares with the other of the two random crops into a single, larger, patch.

6. The computer-implemented method of claim 5, further comprising executing a local decomposition branch that enforces the model to learn fine-grained anatomical details in a whole-to-part manner.

7. The computer-implemented method of claim 6, wherein executing the local decomposition branch that enforces the model to learn fine-grained anatomical details in the whole-to-part manner, comprises decomposing patch embeddings into a plurality of smaller patches.

8. A system comprising:
a memory to store instructions;
a processor to execute the instructions stored in the memory to perform the following operations:
receiving a plurality of unlabeled medical images including consistent global and local anatomical structures;
dividing each of the plurality of unlabeled medical images into a grid of non-overlapping patches;
extracting two random crops from each of the plurality of unlabeled medical images, wherein each of the two random crops comprises a subset of the grid of non-overlapping patches and shares with the other of the two random crops a partially overlapping region of the grid of non-overlapping patches;
executing a global consistency branch that captures discriminative macro-structures in the plurality of unlabeled medical images by extracting global embeddings; and
executing concurrently a local consistency branch that learns fine-grained local anatomical details in the plurality of unlabeled medical images via composition and decomposition operations.

9. The system of claim 8 wherein executing the global consistency branch that captures discriminative macro-structures in the plurality of unlabeled medical images by extracting global embeddings, comprises extracting coarse-grained semantic features of different augmentations within the overlapping regions of each of the plurality of unlabeled medical images.

10. The system of claim 9, wherein extracting coarse-grained semantic features of different augmentations within the overlapping regions of each of the plurality of unlabeled medical images comprises:
resizing the two random crops to a same shape;
adding different augmentations to the resized crops;
feeding the augmented resized crops to a student model and a teacher model to obtain overlapping patch embeddings;
adding an average pooling operator to the overlapping patch embeddings to obtain global embeddings; and
obtaining a plurality of probability distributions by normalizing the global embeddings with a softmax function.

11. The system of claim 8, wherein executing concurrently the local consistency branch that learns fine-grained local anatomical details in the plurality of unlabeled medical images via composition and decomposition operations, comprises integrating the subset of the grid of non-overlapping patches that each of the two random crops from each of the plurality of unlabeled medical images shares with the other of the two random crops into a single, larger, patch.

12. The system of claim 11, wherein executing concurrently a local consistency branch that learns fine-grained local anatomical details in the plurality of unlabeled medical images via composition and decomposition operations further comprises integrating the subset of the grid of non-overlapping patches that each of the two random crops from each of the plurality of unlabeled medical images shares with the other of the two random crops into a single, larger, patch.

13. The system of claim 12, further comprising executing a local decomposition branch that enforces the model to learn fine-grained anatomical details in a whole-to-part manner.

14. The system of claim 13, wherein executing a local decomposition branch that enforces the model to learn fine-grained anatomical details in a whole-to-part manner comprises decomposing patch embeddings into a plurality of smaller patches.

15. A non-transitory computer readable storage media having instructions stored thereupon that, when executed by a system having at least a processor and a memory therein, cause the processor to perform the following operations:
receiving a plurality of unlabeled medical images including consistent global and local anatomical structures;
dividing each of the plurality of unlabeled medical images into a grid of non-overlapping patches;
extracting two random crops from each of the plurality of unlabeled medical images, wherein each of the two random crops comprises a subset of the grid of non-overlapping patches and shares with the other of the two random crops a partially overlapping region of the grid of non-overlapping patches;
executing a global consistency branch that captures discriminative macro-structures in the plurality of unlabeled medical images by extracting global embeddings; and
executing concurrently a local consistency branch that learns fine-grained local anatomical details in the plurality of unlabeled medical images via composition and decomposition operations.

16. The non-transitory computer readable storage media of claim 15 wherein executing the global consistency branch that captures discriminative macro-structures in the plurality of unlabeled medical images by extracting global embeddings, comprises extracting coarse-grained semantic features of different augmentations within the overlapping regions of each of the plurality of unlabeled medical images.

17. The non-transitory computer readable storage media of claim 16, wherein extracting coarse-grained semantic features of different augmentations within the overlapping regions of each of the plurality of unlabeled medical images comprises:
resizing the two random crops to a same shape;
adding different augmentations to the resized crops;
feeding the augmented resized crops to a student model and a teacher model to obtain overlapping patch embeddings;
adding an average pooling operator to the overlapping patch embeddings to obtain global embeddings; and
obtaining a plurality of probability distributions by normalizing the global embeddings with a softmax function.

18. The non-transitory computer readable storage media of claim 15, wherein executing concurrently the local consistency branch that learns fine-grained local anatomical details in the plurality of unlabeled medical images via composition and decomposition operations, comprises integrating the subset of the grid of non-overlapping patches that each of the two random crops from each of the plurality of unlabeled medical images shares with the other of the two random crops into a single, larger, patch.

19. The non-transitory computer readable storage media of claim 18, wherein executing concurrently a local consistency branch that learns fine-grained local anatomical details in the plurality of unlabeled medical images via composition and decomposition operations further comprises integrating the subset of the grid of non-overlapping patches that each of the two random crops from each of the plurality of unlabeled medical images shares with the other of the two random crops into a single, larger, patch.

20. The non-transitory computer readable storage media of claim 19, further comprising executing a local decomposition branch that enforces the model to learn fine-grained anatomical details in a whole-to-part manner.
